(19) United States
(12) Patent Application Publication
(10) Pub. No.: US 2012/0045799 A1
(43) Pub. Date: Feb. 23, 2012

(54) THERMOCYCLER SEAL COMPOSITION, METHOD, AND APPLICATION

(75) Inventor: Peng Zhou, Newtown, PA (US)

(73) Assignee: RHEONIX, INC., Ithaca, NY (US)

(21) Appl. No.: 13/205,028

(22) Filed: Aug. 8, 2011

Related U.S. Application Data
(60) Provisional application No. 61/374,302, filed on Aug. 17, 2010.

(51) Int. Cl. C12P 19/34; C09D 191/06; C12M 1/40

(52) U.S. Cl. 435/91.2; 435/293.1; 106/270

(57) ABSTRACT

An immiscible mixture of wax and silicone oil is provided for application to the inner surface of a thermocycle reactor tube. The mixture is a solid at typical room temperatures and under typical product storage conditions, but at temperatures above the melting point of the mixture (e.g., >46° C. and therefore, at or above 90° C. (which is typical of the first stage of a PCR thermocycle)) the mixture melts and covers the exposed surface of the solution undergoing the thermocycle, thereby sealing the reaction against evaporation and/or condensation over the duration of the multiple thermocycles typical of a PCR reaction. Upon cooling of the reaction tube, the resulting amplicons can then extracted from below the sealing layer.

### RELATED APPLICATION DATA

The instant application claims priority to U.S. provisional application Ser. No. 61/374,302 filed on Aug. 17, 2010, the subject matter of which is incorporated herein by reference in its entirety.

### GOVERNMENT FUNDING

N/A.

### BACKGROUND

1. Field of the Invention

Embodiments of the invention are directed to the field of molecular biology. More particularly, embodiments of the invention are directed to a composition for use in a PCR reaction chamber that reduces and/or prevents fluid evaporation and/or condensation during thermocycling without interfering with the reaction itself; a method for reducing and/or preventing fluid evaporation and/or condensation in a PCR reaction chamber during thermocycling; and applications thereof.

2. Description of Related Art

During a PCR reaction in a reaction vessel, an aqueous solution is repeatedly (e.g., 20-50 times) cycled between a lower temperature of approximately 30° C. to a higher temperature of approximately 98° C. In order to prevent the aqueous solution from losing volume due to evaporation or, e.g., condensation on the side walls of the vessel above the bulk solution, an evaporation or condensation preventative should be used to seal the exposed surface of the solution so that the reaction does not fail due to lack of sufficient solution volume or changes in reaction concentration from an unsealed environment.

To prevent evaporation of the solution or uncontrolled condensation the vessel is typically capped with a physical lid or wax, high purity silicone oil, mineral oil, or some other immiscible substance is typically introduced over the top of the solution. The use of these materials presents certain disadvantages, particularly when used in conjunction with integrated PCR systems where in an integrated system the reaction vessel is not a separate item that can be capped by an operator prior to undergoing thermocycling. For example, mineral oil is a liquid at room temperature and liquid handling of such an oily substance in some types of integrated systems is difficult. Wax is a solid at room temperature though its melting temperature is very controllable, however, wax often impedes certain complex PCR reactions, for example, those reactions using multiple primers for multiplex PCR. High purity silicone oil, like mineral oil, is a liquid at room temperature and has similar handling problems although it does not significantly impede PCR reactions.

For integrated PCR systems it would be advantageous to have a substance that could cover the solution selectively only when, or after, the PCR reaction vessel is filled and conditions for evaporation and/or condensation are initiated.

### SUMMARY

An embodiment of the invention is a composition for use in a PCR reaction vessel (hereinafter referred to as a “tube”) that is a solid in the tube at room temperature and under expected storage temperatures and conditions, and which becomes an immiscible liquid that covers a solution's exposed surface in the tube upon a first rise in temperature during a PCR reaction.

An embodiment of the invention is a composition as mentioned immediately above, in the form of a controlled mixture of high purity silicone oil and wax. The composition is a solid at typical room temperatures and under expected storage conditions (temperature, etc.). The composition is disposed as a layer of solid material on the inside surface of a PCR tube below the opening of the tube; with or without an analyte solution being present in the tube. When the first thermocycle is performed after the analyte is introduced into the tube, the composition melts and covers the surface of the solution. The composition covering the solution thereby prevents evaporation while not impeding the PCR reaction. Upon completion of the reaction, the reacted solution can be removed from underneath the composition by inserting a tube or pipette into the solution and extracting the solution through the tube or pipette leaving the composition in the tube. Alternatively, a lumen may be used to both fill and empty the tube. By placing the bottom end of the lumen close to the bottom of the reactor tube and below the level of the fluid volume to be reacted, the composition still seals the surface of the reaction volume and the lumen can remove most of the fluid from below the composition after the reaction is complete. According to an aspect, the composition consists of a mixture of wax=1% to 20% by volume and the balance silicone oil. According to an aspect, the composition consists of a mixture of approximately 5% wax and 95% silicone oil. The wax may be standard PCR wax (e.g., Sigma Aldrich paraffin wax having a melting point of 58° C.-62° C.). The silicone oil may be standard high purity silicone oil (e.g., Clearco silicone oil 100 CST product code 53148-62-9). Alternatively, any high purity paraffin wax (C₂₀H₄₂-C₄₀H₈₂ with melting points between 46° C.-68° C.) may be used along with any high purity silicone oil. The wax and silicone oil are mixed selectively to produce the desired melting point and handling characteristics of the composition. The greater the proportion of wax the higher the melting point of the composition (though the melting point of the composition will be below that of pure wax i.e. 46° C.-68° C.) and the higher proportion will be less desirable given the potential for inhibition of the PCR reaction by the higher proportion of wax. The lower proportion of wax is therefore more desirable though at the limit of the lower proportion the melting point of the composition is much lower and presents a challenge for maintaining solid form prior to introduction of the solution and initiation of the first thermocycle.

An embodiment of the invention is a method for reducing or preventing evaporation of a solution in a PCR reaction tube and/or undesirable condensation on the inside tube walls during and after a PCR reaction. The method includes the steps of coating at least a portion of the inner wall of the PCR tube with a composition comprising, or consisting of, a controlled mixture of silicone oil and wax, in solid form, prior to a PCR reaction. The PCR reaction involves heating the tube, and creating a condition that transforms the composition into an immiscible liquid that floats on the surface of a solution in the tube and thereby seals the solution's surface in the tube to prevent evaporation and/or condensation. In an aspect, the condition that transforms the composition into a liquid is (but is not limited to) a thermocycling step of a PCR process. In an aspect, the method involves coating at least a portion of the inner wall of the tube with mixture of 1% to 20% by volume wax and the balance silicone oil. In an aspect, the method involves using a mixture of approximately 5% wax and 95% silicone oil. In an aspect, the method further involves extracting the solution whose exposed surface is covered by the layer of the immiscible composition.

All embodiments and aspects of the invention are particularly applicable to microfluidic systems, methods, and applications.

### BRIEF DESCRIPTION OF THE DRAWINGS

FIG. 1A is a cross sectional view of an amplification reactor (tube) showing a fluid volume covered by a layer of an immiscible wax/silicone oil composition, according to an illustrative embodiment of the invention;

FIG. 1B is a cross sectional view of an amplification reactor (tube) showing a solidified layer of the wax/silicon oil composition after the fluid volume was removed from the tube, according to an illustrative embodiment of the invention;

FIG. 2 is a cross sectional view of an empty amplification reactor (tube) showing a solid wax/silicone oil composition disposed on a portion of the inner surface of the tube, according to an illustrative embodiment of the invention;

FIG. 3A is a cross sectional view of an amplification reactor (tube), showing a solid wax/silicone oil composition disposed on a portion of the inner surface of the tube (including a reaction solution), inserted into a heater prior to the initial thermocycle, according to an illustrative embodiment of the invention;

FIG. 3B is a cross sectional view of an amplification reactor (tube) inserted into a heater showing a layer of the wax/silicon oil composition after the initial thermocycle, according to an illustrative embodiment of the invention; and

FIG. 4A is a cross sectional view of an alternative arrangement of an amplification reactor (tube), showing a solid wax/silicone oil composition disposed on a portion of the inner surface of the tube, inserted into its heater prior to introducing a fluid into the tube and prior to the initial thermocycle, where the tube also includes a lumen for introduction of the fluid into the tube and for extraction of the fluid from underneath the immiscible composition layer after the reaction is complete, according to an illustrative embodiment of the invention:

FIG. 4B is a cross sectional view of an alternative arrangement of an amplification reactor (tube), showing a solid wax/silicone oil composition disposed as a ring on a portion of the inner surface of the tube, inserted into its heater prior to introducing a fluid into the tube and prior to the initial thermocycle, where the tube also includes a lumen for introduction of the fluid into the tube and for extraction of the fluid from underneath the immiscible composition layer after the reaction is complete, according to an illustrative embodiment of the invention;

FIG. 4C is a cross sectional view of an alternative arrangement of an amplification reactor (tube) inserted into a heater showing a lumen and showing the solidified layer of the wax/silicon oil composition after the fluid volume was removed from the tube, according to an illustrative embodiment of the invention.

### DETAILED DESCRIPTION OF THE INVENTION

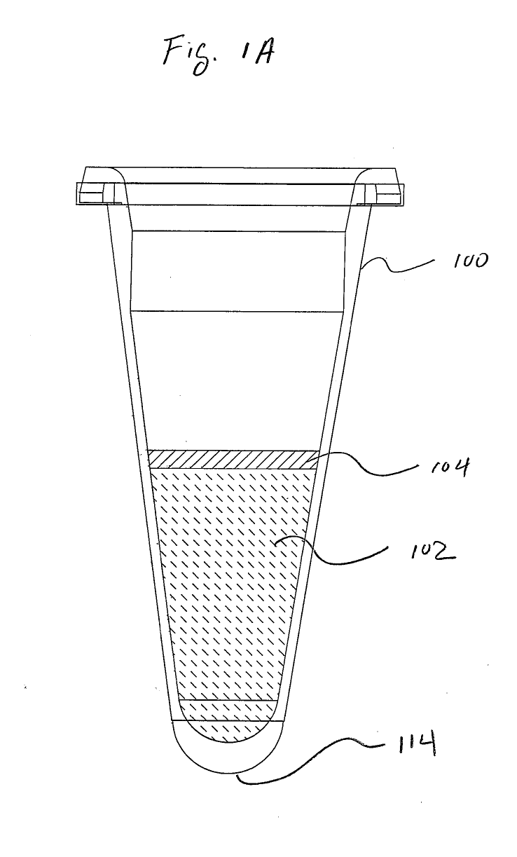

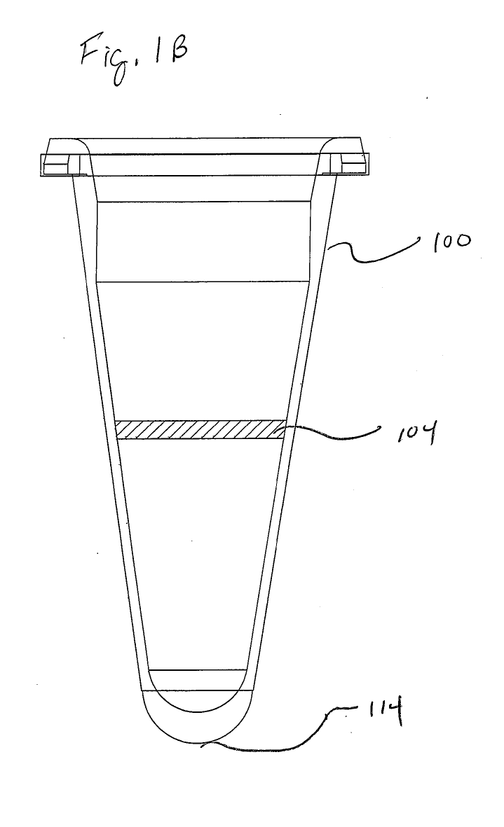

As shown in FIG. 1A, a typical PCR reaction tube 100 is a cone-shaped ampule that tapers to a closed end 114. Purified nucleic acids and a PCR master mix (hereinafter, the “solution”) 102 are introduced into the tube and subjected to variable (PCR) heating and cooling cycles (thermocycles). The first such PCR cycle is always an extended temperature cycle above 90° C. The temperature then cycles repeatedly from about 30° C. to about 98° C. until 20 to 50 such cycles have been completed. During the process, the small volume of solution 102 in the tube is subject to evaporation due to the elevated temperatures and, condensation may occur as the system is cooled. If the reaction is open to the atmosphere, then the small volume of solution will undergo changes in concentration of the reactants with a resulting variability or complete failure of the reaction. As shown in FIG. 1A, a layer of an immiscible, liquid silicone oil (95%)/wax (5%) mixture 104 floats on the surface of the solution 102 and thereby prevents the solution from evaporating and, at the same time prevents any condensation from entering the reaction volume. Given the geometric shape of the tube 100 as an inverted cone, the immiscible, liquefied wax/silicone oil composition, upon re-solidifying, will be at the height of the solution's surface when the reactor tube is cooled. Therefore, the wax/silicone oil composition entirely seals the reaction and remains separate from the reaction for the entire PCR process.

As shown in FIG. 1B the solidified wax/silicone oil composition 104 remains in the tube after solution 102 is removed.

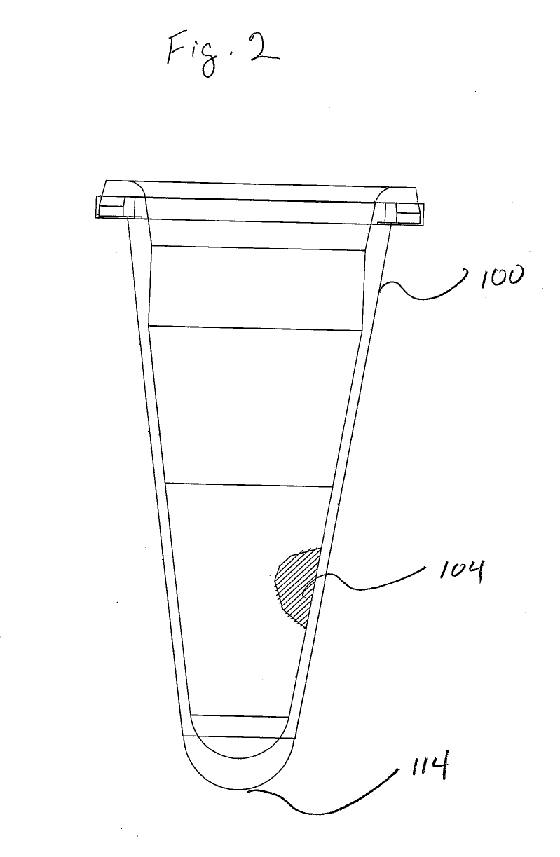

As shown in FIG. 2, a portion of an amplification reactor tube's inner surface is coated with a solid layer of the wax-silicone oil composition 104 in a region below the opening of the tube. The solid composition 104 may be coated nearer the opening or nearer the bottom of the tube depending upon the desired solution volume of the reaction. The layer will generally be advantageously applied lower within the tube when a smaller reaction volume is used. The layer will advantageously be partially or entirely submerged in the reaction volume.

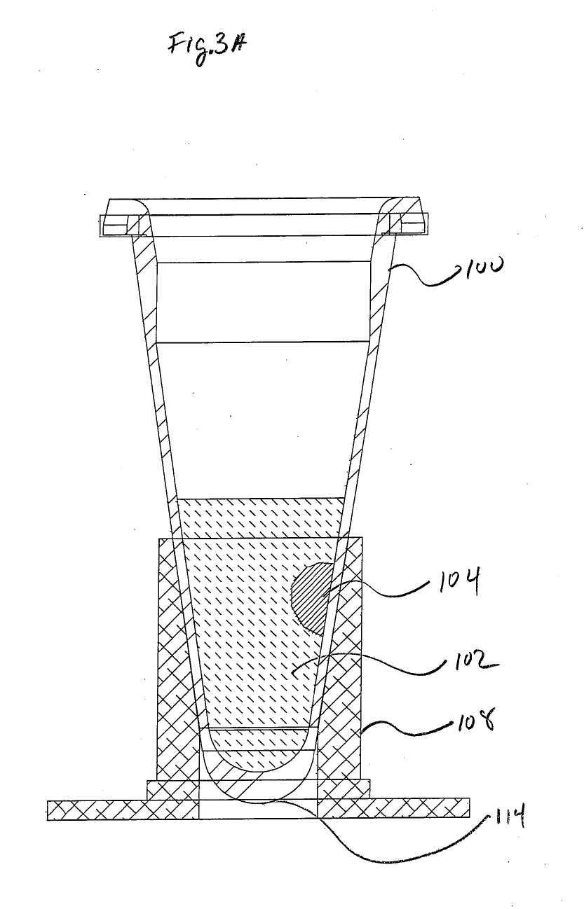

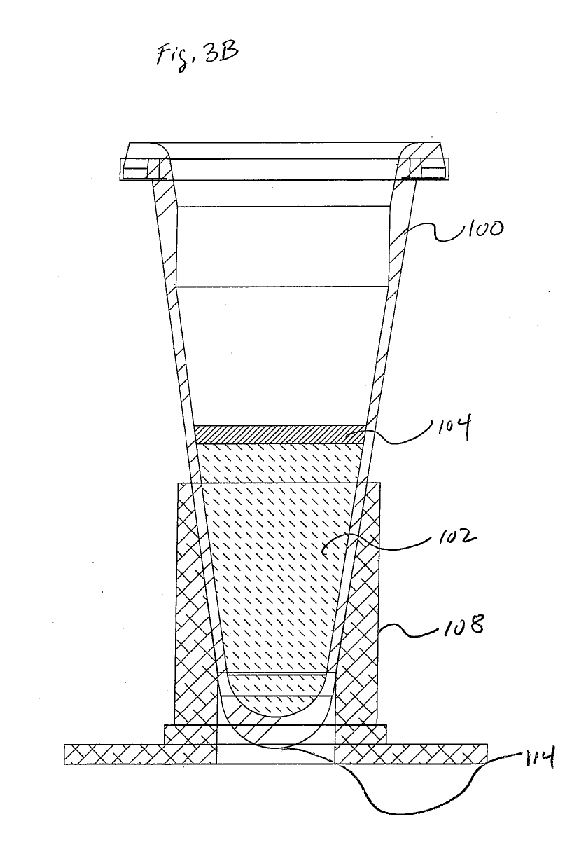

As shown in FIG. 3A, the tube 100 containing the wax/silicone oil composition 104 is filled with the desired amount of analyte solution and placed into the thermocycler (heater) 108. The initial thermocycle is typically an extended cycle above 90° C. The wax-silicone oil composition is designed to melt below the temperature of the initial thermocycle and, since it is both immiscible and lower in density than the reaction solution, as shown in FIG. 3B, it melts and covers the surface of the reaction solution.

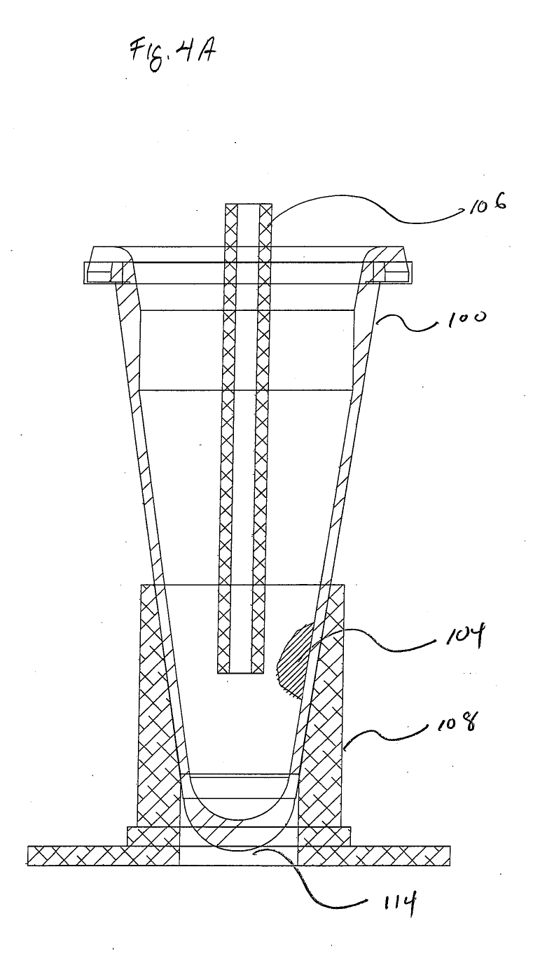

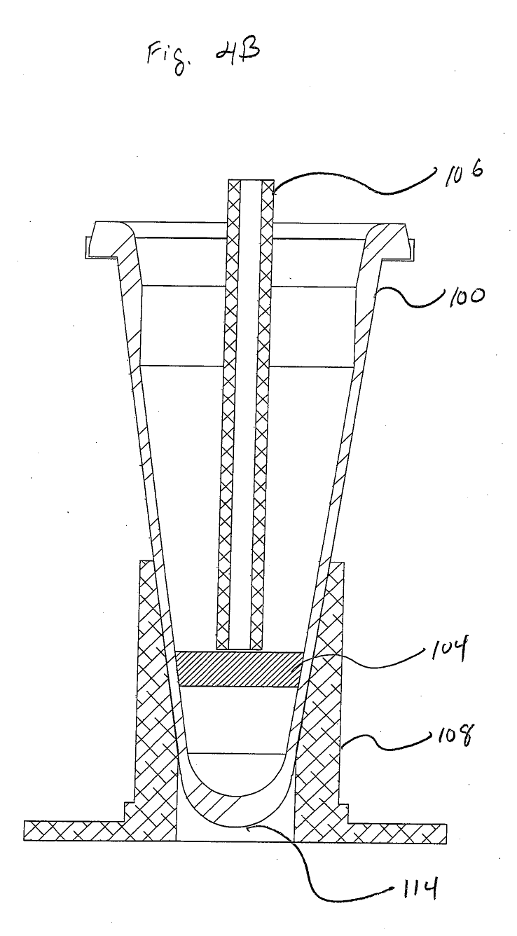

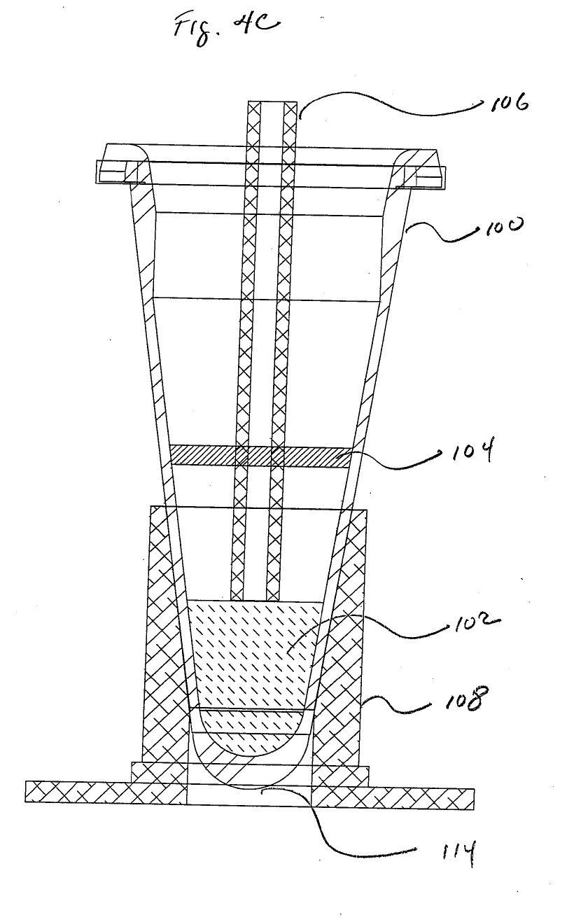

As shown in FIG. 4A, the reactor tube 100 may be penetrated by a lumen or a pipette tip 106 for introduction of the solution prior to the first thermocycle or extraction of the solution from underneath the wax/silicone oil composition 104 upon completion of the reaction.

As shown in FIG. 4B the wax/silicone oil composition may be deposited as a ring around a portion of the inner surface of the tube.

As shown in FIG. 4C as an illustrative example, the solution in the tube is cooled below the melting point of the wax/silicone oil composition leaving the layer hardened and suspended from the tapered inner wall of the tube. In an alternative manner, the lumen 106 may be used both to fill and to empty the tube and may remain in place throughout the reaction. In the case where the lumen remains in place, the bottom of the lumen will advantageously be placed close to the bottom of the tube to avoid any evaporation through the lumen during the reaction. The lumen may also be capped to further avoid evaporation.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening.

The recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not impose a limitation on the scope of the invention unless otherwise claimed.

No language in the specification should be construed as indicating any non-claimed element as essential to the practice of the invention.

It will be apparent to those skilled in the art that various modifications and variations can be made to the present invention without departing from the spirit and scope of the invention. There is no intention to limit the invention to the specific form or forms disclosed, but on the contrary, the intention is to cover all modifications, alternative constructions, and equivalents falling within the spirit and scope of the invention, as defined in the appended claims. Thus, it is intended that the present invention cover the modifications and variations of this invention provided they come within the scope of the appended claims and their equivalents.

I claim:

1. A composition, comprising:
a mixture of wax and silicone oil that is in a solid phase at a temperature less than about 46° C. and in a liquid phase at a temperature greater than about 46° C., wherein the mixture is from 1% to 20% wax and 99% to 80% silicone oil.

2. The composition of claim 1, wherein the mixture is from 1% to 5% wax and 99% to 95% silicone oil.

3. The composition of claim 1, consisting of a mixture of wax and silicone oil.

4. The composition of claim 3, wherein the mixture is from 1% to 5% wax and 99% to 95% silicone oil.

5. A PCR tube, comprising:
a solidified mixture of wax and silicone oil disposed on at least a portion of an inner surface of the tube.

6. The PCR tube of claim 5, wherein the mixture is from 1% to 20% wax and 99% to 80% silicone oil.

7. The PCR tube of claim 6, wherein the mixture consists of wax and silicone oil.

8. The PCR tube of claim 6, wherein the mixture is from 1% to 5% wax and 99% to 95% silicone oil.

9. A method for reducing or preventing evaporation of a solution in a PCR reaction tube and/or undesirable condensation on the inside tube walls during and after a PCR reaction, comprising:
providing a PCR tube having a coating on at least a portion of an inner wall surface of the PCR tube of a composition of silicone oil and wax in solid form;
providing a solution in the tube that will undergo a PCR reaction; and
creating a condition that transforms the solid composition into a liquid that covers the surface of the solution.

10. The method of claim 9, further comprising at least partially filling the PCR tube with a solution prior to the transformation step.

11. The method of claim 9, wherein the step of creating a condition that transforms the solid composition into a liquid composition further comprises heating the tube to at least a melting temperature of the composition.

12. The method of claim 9, further comprising providing the PCR tube coated with a mixture of from 1% to 20% wax and 99% to 80% silicone oil.

13. A composition, comprising a mixture of wax and silicone oil that is a solid at a temperature less than about 46° C. and a liquid at a temperature greater than about 46° C.
